(19) United States
(12) Patent Application Publication
(10) Pub. No.: US 2016/0250127 A1
(43) Pub. Date: Sep. 1, 2016

(54) USE OF A POLYOL IN THE REMINERALISATION OF ENAMEL

(71) Applicant: ROQUETTE FRERES, Lestrem (FR)

(72) Inventors: Laetitia GUERIN-DEREMAUX, NIEPPE (FR); Clementine THABUIS, LEDRINGHEM (FR); Daniel WILS, MORBECQUE (FR); Bertrand RODRIGUEZ, LAMBERSART (FR); Antoine BARRE, SAINT ANDRE LEZ LILLE (FR)

(21) Appl. No.: 15/027,890

(22) PCT Filed: Oct. 9, 2014
(86) PCT No.: PCT/FR2014/052568

§ 371 Date: Apr. 7, 2016

(30) Foreign Application Priority Data
Oct. 9, 2013 (FR) 1359812

(51) Int. Cl. A61K 8/60; A23G 4/10; A23G 3/42; A61Q 11/00

(52) U.S. Cl. CPC A61K 8/60; A61Q 11/00; A23G 4/10; A23G 3/42; A61K 2800/30; A23V 2002/00

(57) ABSTRACT

A composition including at least a polyol for the prevention of oral and dental infections or diseases and/or maintaining good oral and dental hygiene, thus allowing the maintenance of good oral and dental health. More specifically, treating demineralisation of dental enamel, and especially for stimulating remineralisation thereof, using the composition including at least one polyol selected from maltitol and xylitol.

### FIELD OF THE INVENTION

The present invention relates to a composition comprising at least one polyol, intended for the prevention of oral and dental infections or diseases and/or for maintaining good oral and dental hygiene, consequently allowing the maintenance of good oral and dental health. The present invention relates more particularly to the use of a composition comprising at least one polyol chosen from maltitol and xylitol for treating the demineralization of dental enamel, and especially for stimulating the remineralization thereof.

### TECHNOLOGICAL BACKGROUND

Dental health concerns all aspects of the health and operation of our mouth, in particular the teeth and the gums. In addition to allowing us to eat, speak and laugh, the teeth and the gums must also combat infection, which infection can cause tooth decay, gum inflammation, tooth loss and bad breath.

There are four major mechanisms of tooth wear:
attrition, which is wear resulting from the rubbing of the teeth together during normal operation or during pathological habits such as grinding of the teeth (bruxism).
Abrasion, which results from mechanical wear caused by the rubbing of a substance other than the teeth.
Abfraction, which is caused indirectly by bruxism and jaw-clenching habits and which contributes to “dissolving” the enamel crystals. This phenomenon causes a loss of enamel at the neck of the teeth close to the gums.
Erosion, which is a loss of mineral substance of the enamel and of the dentine caused by a chemical process. One of the main causes is diet. More specifically, the consumption of acidic drinks and foods promotes this erosion which results in the formation of caries.

Tooth decay is a transmissible infectious disease. It is in particular the result of the acid erosion generated by dental plaque bacteria. Some bacteria, such as Streptococcus mutans and certain lactobacilli, can be transmitted, for example, from parents to their children. These bacteria are cariogenic, which means that they facilitate tooth degeneration. They form a sticky film, more commonly known as dental plaque, at the surface of the tooth. Each day, generally after meals, bacterial plaque forms rapidly on the teeth and constitutes a thin and tacky matrix, also called biofilm, at the surface of the enamel of the teeth and on the gums. This matrix encompasses food debris and bacteria which develop by feeding on the fermentable carbohydrates originating from foods and drinks. The acids produced by the bacteria dissolve the minerals such as calcium and phosphorus of the tooth. This is referred to as demineralization. This demineralization constitutes the starting point of the decay; it is stage 1 for which there is no sensitivity.

Stage 2 is the involvement of the dentine (substance forming the internal layer of the tooth) recognizable by sensitivity to external stimuli such as hot, cold or sugar. Stage 3 is characterized by such hard tissue destruction that there is involvement of the dental pulp characterizable by spontaneous pain (toothache). Finally, stage 4 is pulp mortification or spontaneous devitalization with bacterial proliferation in the canals and around the tooth. This is the most serious stage leading to dental infection or abscess. This seat of infection represents a danger to general health. The bacteria can migrate into the body by the blood and graft themselves onto organs such as the heart, the kidneys, the joints, etc. Decay is an evolving process and there cannot be a spontaneous recovery.

However, tooth decay is not inevitable: it is possible to combat it. Saliva eliminates food debris from the mouth, neutralizes the acids produced by plaque bacteria and supplies the calcium and phosphorus required by the teeth in a process called “remineralization”. Saliva also acts as a reservoir for the fluoride from toothpaste or from fluoridated water. Fluoride helps to control tooth decay by remineralizing the teeth and by inhibiting bacterial production of acids, thereby reducing or slowing down the degradation process.

Tooth decay occurs only once the equilibrium is broken in the long term between the process of demineralization and remineralization of the tooth.

Dental enamel is a strongly mineralized tissue. It is in fact made up of 96% of inorganic materials and 3% of an organic matrix (enamel proteins, enamelin and lipids), the remaining 1% being water. Enamel is therefore a very compact, weakly permeable structure. However, since it has been observed that the tissue allows the passage of liquids composed of small ions or of organic colorants, it is assumed that a system of pores, also accessible to more bulky molecules, is present. Although water is present in a small amount, it is thought that the latter allows the formation of a hydrating layer around the crystals of dental enamel, said layer being required for ion exchange and the penetration of molecules into the dental surface. The presence of water inside these micropores is thought to constitute the principal mode of diffusion allowing certain elements, such as bacterial acids, fluoride (F⁻) or calcium phosphate (CaPO₄), to be introduced into the surfaces. The organic acids produced by the plaque microorganisms can diffuse due to the presence of water in the pores at the surface of the enamel and thus come into direct contact with the crystalline structures of the tooth. Thus, they dissolve them, acting from the inside, and release the minerals that they contain and more particularly the calcium phosphate. This process is called demineralization.

It should not be forgotten that the solubility of calcium phosphate present in the biological structures increases considerably when the pH decreases. This explains the importance of the pH of the saliva and of the plaque in the health of the oral cavity. A decrease in pH leads to dissolution of the crystalline structures, while an increase causes precipitation for mineral components. This leads to what are referred to as initial carious lesions.

It is a carious lesion without cavitation which is not characterized by a loss of tissue, but by an initial demineralization clinically visible by the presence of a typically chalky white spot on the surface of a tooth. This initial lesion is characterized, however, by dissolution of the hard tissues of the subsurface layer, without involvement of the external layer of enamel and, in principle, without particular modification of the morphology of the tooth surface. Given the initial nature of the lesions, it is possible to reverse the demineralization process by intervening on the lesions, such as the white lesions, by stimulating and guiding a remineralization process.

As has been indicated for the “demineralization” process, each of the components of the crystalline structures of the enamel spreads to the exterior once dissolved. On the other hand, in the case of “remineralization”, the reverse procedure can be observed: the mineral component diffuses from the saliva or the plaque, passing through the organic network of the tissue, until it comes into contact with the damaged crystalline structures of the enamel that will thus be repaired. However, the term “remineralization” does not indicate a complete reconstruction of the damaged tissues (restituo ad integrum), but the redeposition of the crystalline structures inside a partially damaged tissue. In reality, in the initial carious lesions, it is never possible to carry out a pure and simple regeneration process, but rather an overall repair that will never be identical in the slightest details to the structures damaged by the carious process. Consequently, it is possible to speak of remineralization from the “macroscopic” point of view, with restoration of enamel resistance, reduction of the chalky appearance and repair of the initial carious lesion, but not from the “microscopic” point of view. The lesion repaired in the context of a remineralization process can be compared to a “scar”.

Currently, the two most effective solutions for remineralizing and “curing” the initial carious lesions are fluoride-containing products and derivatives of milk casein containing amorphous calcium and phosphate (CPP-ACP for casein phosphopeptide-amorphous calcium phosphate).

First of all, fluoride-containing products are classified in two main categories: products intended for the general public and products intended for professionals. The products intended for the general public are on free sale in pharmacies and department stores and are aimed at consumers. However, they are confronted with considerable restrictions regarding the fluoride concentration which, in many European countries, cannot exceed 1500 ppm. Their remineralizing capacity is not very powerful, but they are prophylactic devices that are valid in the context of the prevention of carious lesions that have not yet occurred. The most widespread products are toothpaste, throat sprays and oral gels. The products intended for professionals are, for their part, reserved for sale to specialists (physicians, dentists, pediatricians) or can be sold only on medical prescription. The fluoride concentration in these products can greatly exceed 100 000 ppm. They are very representative of the products intended for remineralizing treatments and are sold in the form of varnishes (from 2000 to 32 000 ppm), of neutral or slightly acidic gels (from 12 500 to 20 000 ppm) or of liquids. They must all generally be applied regularly (ideally every three months) to the damaged dental surfaces.

Fluoride has a Triple Action: remineralization stimulant, demineralization inhibitor and stabilizer of salivary pH around physiological values. The synergy of these three functions explains the strong preventive and repair action of fluoride when it is in contact with the teeth.

With regard to remineralization, it also appears that, in the absence of fluoride and in the presence of a particular pH, calcium phosphates (CaPO₄) can precipitate again on the surface of the teeth, forming mainly “brushite”, which is not a natural component of the tooth and which is much less solid than hydroxyapatite. In the presence of fluoride, the latter will be integrated into calcium and phosphate during a remineralization phase in the form of a fluoroapatite which, as previously indicated, is a component which is much more resistant to acid attacks than hydroxyapatite and less soluble. Furthermore, in the presence of saliva supersaturated with calcium, with phosphate and with fluoride, a part of the crystalline structure of the apatite will tend to increase by accumulation of successive deposits. This phenomenon explains the remineralization processes that can be carried out in the white lesions through the use of certain techniques and of certain products which allow repair and also reformation of the partially damaged crystals.

Ionomer glass cements constitute another advantageous form of remineralization induced by fluoride since they release fluoride after application, which can lead to a repair phenomenon at the surfaces and allow the latter, subsequently, to receive long-term reconstruction materials. This phenomenon typical of ionomer glass cements earns them the name of “bioactive materials”.

Moreover, over the past few years, milk casein derivatives containing in particular amorphous calcium and phosphate in high concentrations (CPP-ACP, recaldent) have also contributed a great deal to dental structure remineralization. These products have the property of supersaturating the dental environment by means of high concentrations of calcium and of phosphate, so that these chemical elements can penetrate into the tooth and be deposited/precipitated therein according to the normal process of osmosis. Since the main components of teeth are calcium and phosphate, the final result will in short be a remineralization of the structures, including of those already damaged. The result is even more obvious if the CPP-ACP is simply accompanied by fluoride (Mi Paste Plus, GC, Europe). For the reasons previously explained, the presence of fluoride is capable of promoting remineralization through the predominant formation of fluoroapatite, which prevents the deposit of high concentrations of calcium and of phosphate of the CPP-ACP, responsible for large amounts of brushite. Although the fluoride-based varnishes or gels can be administered every two to four months, CPP-ACP-based products must be applied more frequently in order to obtain more marked results. CPP-ACP is a cow's milk casein derivative which, contrary to fluoride, is not a product that is potentially dangerous at high concentrations. This application frequency does not therefore pose a problem and CPP-ACP can be prescribed without problem to patients who will themselves be able to manage the applications. The only limits exhibited by CPP-ACP lie in possible allergies to milk proteins (relatively frequent) or to any other component (more rare).

There are many methods for using CPP-ACP:
1) Single application by the dentist or the hygienist after dental hygiene care. Ideal in particular for remineralizing surfaces which have been scaled and for treating the slight dentine hypersensitivities which generally occur after prophylactic care.
2) Replacement or supplementation of normal toothpaste with a treatment with makes provision for more applications at home. Ideal for strong hypersensitivities, for daily protection of patients strongly exposed to risks of decay or else for the treatment of small initial carious lesions.
3) Prolonged application, preferably overnight, by means of personalized casts manufactured in the laboratory which protect the product from the washing action of saliva. In this case, the product is kept in contact with the teeth for a long period of time and can offer the maximum remineralization effect. This is the preferred method of use of the formulation of CPP-ACP with fluoride for the treatment of carious lesions without cavitation, even those which are widespread.

Fluoride and CPP-ACP currently constitute the most effective principles for obtaining dental remineralization without having recourse to an invasive procedure. They can represent the sole treatment for resolving initial carious lesions if they are used very early on said lesions. If the lesions become cavity lesions, it will no doubt be necessary to intervene by means of a more invasive technique, but the joint use of products based on fluoride or on CPP-ACP can also prove to be useful for partially remineralizing dental tissues and limiting the implementation of invasive procedures to non-remineralizable tissues only.

However, these two methods, these two solutions (fluoride and CPP-ACP), also have major disadvantages in terms of their use.

First of all, an excess and an accumulation of fluoride in the body can cause harmful effects on health and lead to dental fluorosis. The WHO has set a fluoride dose not to be exceeded in order to avoid any risk of fluorosis, said dose being 0.05 mg/d per kilogram of body weight without exceeding 1 mg/d. Dental fluorosis is a pathological condition which results from an excessive intake of fluoride, essentially during the tooth development period. Excessive ingestive fluoride concentrations disturb the functioning of the cells responsible for enamel formation. These cells, called ameloblasts, do not produce enamel correctly in the presence of fluoride. The seriousness of dental fluorosis ranges from very slight to serious depending on the magnitude of the exposure to fluoride during the tooth development period. The stronger the fluoride poisoning was during the tooth mineralizing phase, the more visible the symptoms will be. They range from a few small white spots to brown mottling on the tooth enamel. These effects are irreversible. However, a dentist can conceal them using collages of materials on the marked teeth.

The dangers of excess fluoride may not stop there. Dental fluorosis can be followed by skeletal fluorosis. This is a disease which results from the excessive accumulation of fluoride in the bones, causing changes in the structure of the bones and making them extremely fragile and brittle. The first stages of skeletal fluorosis are characterized by an increased bone mass detectable with X-rays. If the fluoride intake remains very high over the course of many years, the changes in the bones can lead to stiffness and pain in the joints. The most severe form of skeletal fluorosis is known as “crippling skeletal fluorosis” and can result in ligament calcification, immobility, muscle loss and also neurological problems associated with spinal cord compression.

In fact, fluoride may enter the brain and pass through the various tissues of the body. It may cause intellectual problems (loss of will-power, mental retardation) and phychiatric problems (violence, addictions, insanity). It acts deeply by promoting accelerated degeneration of the body regardless of age.

The use of CPP-ACP must be done with monitoring by a dental professional, which can make its use complicated. Furthermore, not all individuals affected by enamel demineralization problems necessarily have the means to be monitored regularly by a dentist in order to remedy this. The treatment is lengthy and restrictive. Finally, CPP-ACP is a milk casein derivative and cannot therefore be used by individuals who suffer from milk protein allergies.

Thus, the efficacy, the absorption, the simplicity of use, the side effects, the regulation and the cost associated with the treatments that already exist are all problems which need to be overcome at the current time.

There is therefore at the current time still a real need for an inexpensive, easy-to-use solution which makes it possible to remineralize dental enamel, and which is intended for preventing oral and dental infections or diseases and/or maintaining good oral and dental hygiene, consequently making it possible to maintain good oral and dental health.

### SUMMARY OF THE INVENTION

Armed with this observation and after numerous research studies, the applicant company has to its credit met all the demands required and has found that such an objective can be achieved as long as a composition comprising a polyol is used. The invention relates to a composition comprising at least one polyol, preferably chosen from maltitol and/or xylitol, for use in the treatment or prevention of dental enamel demineralization.

More specifically, the invention relates to a composition comprising at least one polyol, preferably chosen from maltitol and/or xylitol, for the treatment of dental enamel demineralization.

According to the invention, said composition is administered, in humans or in animals, in a proportion of from 0.05 to 3 g/kg/day, preferentially from 0.1 to 2.5 g/kg/day, even more preferentially from 0.2 to 2 g/kg/day, preferentially for 3 months and even more preferentially for 6 months.

According to another preferential mode, the composition is characterized in that it is in the form of a sugar-free confectionery, said sugar-free confectionery being selected from the group consisting of hard boiled candies, dragees, jelly candies, gums, caramels, toffees and fudges, tablets, lozenges, marshmallows, chewing gums, bubble gums and chewy pastes.

One particularly advantageous mode of the invention relates to a composition in the form of a sugar-free chewing gum.

Finally, the invention relates to a method for treating demineralization, consisting in administering at least one polyol, preferably chosen from maltitol and xylitol, in the form of a sugar-free confectionery, in a proportion of from 3 to 5 daily intakes of two confectioneries per intake, each confectionery containing between 0.5 and 1 g of polyol.

Said method for treating demineralization is characterized in that the confectionery employed is preferably a chewing gum.

One advantageous mode of the invention relates to a method for treating demineralization, consisting in carrying out 5 daily intakes of two chewing gums per intake, each chewing gum containing between 0.5 and 1 g of polyol, preferably maltitol.

The final aspect of the invention relates to the use of an effective amount of a polyol for preparing a medicament intended for dental enamel remineralization.

### DETAILED DESCRIPTION OF THE EMBODIMENTS

Thus, the present invention relates to a composition comprising at least one polyol for use in the treatment or prevention of dental enamel demineralization.

More specifically, the present invention relates to a composition comprising at least one polyol for use in dental enamel remineralization.

In the present invention, the name “dental enamel” used should be understood in its broadest interpretation and as denoting the external visible part of the crown of the teeth, which protects the various internal layers such as the dentine and the pulp.

In the present invention, the term “polyols” denotes the products obtained by catalytic hydrogenation of monosaccharide or disaccharide reducing sugars. In particular, the polyol can be chosen from the group comprising sorbitol, xylitol, erythritol, maltitol, isomalt, isomaltitol, lactitol, alpha-D-glucopyranosyl-1,6-sorbitol (=1,6-GPS), alpha-D-glucopyranosyl-1,1-mannitol (=1,1-GPM), alpha-D-glucopyranosyl-1,1-sorbitol (=1,1-GPS) and any mixtures thereof, and preferably from maltitol, xylitol, sorbitol type 20/60, isomalt type M or erythritol.

According to one preferential mode, the composition according to the invention is characterized in that the polyol is chosen from maltitol or xylitol. More preferentially, the polyol is maltitol.

The applicant company has for many years been involved in research on oral hygiene. Its numerous research projects on the beneficial properties of polyols have promoted the growth of the latter in various “sugar-free” food types.

Polyols are Unquestionably Toothfriendly: they are non-fermentable by the oral flora, non-acidogenic, and non-cariogenic, and they create none of the conditions favorable to the occurrence of decay. On the contrary, by limiting the action of bacteria that are harmful to the teeth and the development of dental plaque, polyols prevent dental enamel demineralization. These are all properties which allow them to proudly sport the logo of the dental association Toothfriendly International. Polyol-based products can therefore also meet the criteria required to display this logo if they are free of cariogenic or erosive potential sometimes provided by other ingredients.

In the present invention, the term “non-cariogenic” is intended to mean compositions comprising a polyol and which do not induce caries when they are consumed.

More specifically, these compositions lead to less acidification by the bacteria in the mouth than compositions containing conventional sugars such as sucrose, glucose or fructose.

The non-cariogenic effect is in fact due to the presence, in the oral cavity, of a large number and of a large variety of bacteria, in particular cariogenic bacteria (mutant streptococci in particular) which colonize the dental plaque (or dental film) and metabolize and ferment food sugars, leading to the production of acids, in particular of lactic acid. The latter allow a decrease in the peripheral pH of the tooth below the fateful pH of 5.7, which has the effect of dissolving the hydroxyapatite of the dental enamel and of creating cavities therein. The tooth is then weakened since the high acidity causes demineralization (dissolution) of the dental enamel. The carie then progresses inside the tooth and reaches the pulp, causing pain.

Indeed, repeated consumption and also a long residence time in the mouth of foods rich in fermentable carbohydrates (containing sugar or sucrose, fructose, starch, etc.) form an environment conducive to the development of caries.

Polyols are widely used in the replacement of sugar, and in particular in confectioneries, owing to their low-calorie but especially non-cariogenic nature. More recently, the beneficial effect to health of polyols has been widely acknowledged owing to their hypoglycemic and hypoinsulinemic nature. Polyols therefore potentially have a beneficial effect in the context of cardiovascular diseases or of diabetes, but also in the context of obesity-associated diseases.

It has therefore been known for a long time that the consumption of polyols, in particular in confectioneries, does not induce caries by not promoting dental enamel demineralization.

Today, the applicant company goes even further in its assertions. Indeed, its numerous research studies have made it possible to demonstrate that, in addition to the fact that it does not cause enamel demineralization inducing the formation of caries, the use of a composition comprising a polyol, and more particularly maltitol or xylitol, also enables enamel remineralization when the latter is damaged.

These results are all the more surprising with regard to the use of a composition comprising maltitol.

In the present invention, the enamel remineralization is measured using the QLF™ (Quantitative Laser-induced Fluorescence) quantitative method which quantifies loss of fluorescence of tissues which contain caries (compared with the natural fluorescence of healthy dental tissues), and therefore which, conversely, makes it possible to quantify the gain in fluorescence of remineralized dental tissues compared with their initial state which exhibited less fluorescence due to demineralization.

The detailed principle of this method of analysis will be developed hereinafter.

Thus, the present invention makes it possible to obtain an increase in the fluorescence levels obtained with measurements according to the QLF™ method. This increase therefore reflects a remineralization of the enamel and more particularly a positive action on the “primary” caries, also called initial carious lesions.

According to the present invention, said composition is characterized in that it is administered, in humans or in animals, in a proportion of from 0.05 to 3 g/kg/day, preferentially from 0.1 to 2.5 g/kg/day, even more preferentially from 0.2 to 2 g/kg/day. Preferably, said composition according to the invention is characterized in that it is suitable for administration, in humans or in animals, in a proportion of from 0.05 to 3 g/kg/day, preferentially from 0.1 to 2.5 g/kg/day, even more preferentially from 0.2 to 2 g/kg/day, preferentially for 3 months and even more preferentially for 6 months. According to the present invention, the composition containing a polyol, preferably chosen from maltitol and xylitol, is in the form of a sugar-free confectionery.

In the present invention, the term “confectionery” (synonyms: candies, sweets, sweet things, etc.) denotes any flavored food product which has a sweet flavor, the consistency of which may be hard or soft, which may be coated with chocolate, which is consumed by sucking and/or by chewing in the oral cavity and which does not contain sugar.

According to one preferential mode, the confectioneries of the present invention are all the confectioneries of hard boiled candy (more commonly called hard candy), dragee, jelly candy, gum, caramel, toffee and fudge, chewing gum, bubble gum, chewing paste, tablet or lozenge type.

According to another preferential mode, the confectioneries of the present invention may be aerated confectioneries, for example marshmallows.

According to one variant of the invention, the confectioneries of the present invention may be film-coated. The film coating consists of the application of a film-forming liquid composition which, after drying, becomes a protective film. This film coating serves, for example, to protect the active ingredients contained in the confectionery, to protect the confectionery itself against moisture, impacts, friability, and also to confer on the confectioneries attractive visual properties: shine, uniform color, smooth surface, etc.

According to an even more preferred mode of the invention, the composition containing a polyol, preferably chosen from maltitol and xylitol, is in the form of a sugar-free chewing gum.

In the present invention, the term “chewing gum” is used without distinction to denote chewing gums and bubble gums, the difference between these two types being, moreover, quite vague. It is customary to say that chewing gums are chewed, whereas bubble gums are intended for making bubbles, and are thus conventionally mostly consumed by young consumers.

Most chewing gums, whether they are with or without sugar, and sweet-coated or not, essentially comprise a water-insoluble gumbase, hydrosoluble sweetening agents provided in liquid and/or pulverulent form and flavorings. They often comprise other ingredients such as dyes, emulsifiers, plasticizers, intense sweeteners, water, etc.

The manufacture of sugar-free chewing gum or bubble gum centers, also called tabs, requires the mixing of gumbase with polyols, used as filling sweeteners. Typically, the gumbase represents between 25% and 35% of the centers, and the polyols between 65% and 80%, the rest possibly consisting of flavorings and/or intense sweeteners of aspartame or acesulfame-K type.

The applicant company has, to its credit, demonstrated that a composition comprising at least one polyol allows remineralization of the dental enamel and therefore a curative treatment of the “initial” carious lesions.

According to an even more preferential mode of the present invention, said confectionery is a polyol-based, sugar-free chewing gum which may or may not be sweet-coated and/or film-coated by means of one of the known and described methods of the prior art.

Without being limited to a particular mechanism, the consumption of a chewing gum according to the invention is capable of contributing to the maintaining of good dental hygiene, by preventing, inter alia, the formation of dental plaque, responsible for many oral and dental infections or diseases. The chewing of said chewing gum promotes activation of the defense mechanisms of the saliva, thus preventing bacterial growth at the surface of the teeth. This beneficial effect is also found when the polyol is included in a confectionery to be sucked or chewed. Once again, saliva production is promoted, thereby making it possible to considerably reduce bacterial growth on the teeth.

According to another preferential mode of the present invention, the amount of confectionery consumed per day and per individual is between 0.5 g and 50 grams. According to an even more preferential mode, the amount consumed per day is between 2 g and 30 grams.

In the particular case where the confectionery is a chewing gum or a chewy paste, the amount of confectionery consumed per day is less than 25 grams and preferably about 17 grams.

The composition of the present invention can be consumed at any moment judged to be appropriate and for the desired period of time.

According to one preferential mode, the composition is consumed after each ingestion of liquid and/or of food, for instance after meals, times when bacterial attacks and the formation of dental plaque are the most frequent.

According to one preferential mode of the present invention, the composition is consumed at least three times per day, and preferably at least four times per day.

According to another preferential mode, the composition, when it is consumed, remains present in the oral cavity for at least 2 minutes, preferably for at least 3 minutes and even more preferably for at least 5 minutes.

According to an even more preferential mode of the present invention, flavorings such as mint or fruit flavorings can be added in order to promote greater salivary secretion, and thus to reinforce the action of the protection. Other flavorings such as, for example, menthol, eucalyptol, thymol, methyl salicylate, licorice and cinnamic aldehyde can, by virtue of their inherent antibacterial property, reinforce the action of destruction of undesirable microorganisms of the oral cavity.

According to another preferential mode of the present invention, active ingredients can be added to the confectionery. In the present invention, the term “active ingredient” is intended to mean any active molecule which has a demonstrated pharmacological effect and a therapeutic interest which is likewise clinically demonstrated.

For the purposes of the present invention, the term “effective amount” is intended to mean a dose of polyol of about from 0.05 to 3 g/kg/day, preferentially from 0.1 to 2.5 g/kg/day, even more preferentially from 0.2 to 2 g/kg/day.

Advantageously, the pharmaceutical compositions according to the invention also comprise an active ingredient.

The invention is also directed toward a method for treating demineralization, comprising a step of administering, to a subject suffering from dental carious lesions, a pharmaceutically effective amount of a polyol, preferably chosen from maltitol and xylitol, more preferentially maltitol, said polyol being administered alone or as a mixture with at least one additional active ingredient, for treating demineralization and promoting remineralization of dental enamel.

The invention also relates to a method for treating demineralization, consisting in carrying out between 3 and 5 daily intakes of two confectioneries per intake, each confectionery containing between 0.5 and 1 g of polyol, preferably chosen from maltitol and xylitol, more preferentially maltitol.

According to one preferential mode, the invention relates to a method for treating demineralization, consisting in carrying out between 3 and 5 daily intakes of two chewing gums per intake, each chewing gum containing between 0.5 and 1 g of polyol, preferably chosen from maltitol and xylitol, more preferentially maltitol.

According to another preferential mode of the invention, the invention also relates to a method for treating demineralization, consisting in carrying out 5 daily intakes of two chewing gums per intake, each chewing gum containing between 0.5 and 1 g of polyol, preferably chosen from maltitol and xylitol, more preferentially maltitol.

The treatment method according to the invention makes it possible, after six months of application, to visibly remineralize the primary caries in the enamel. This remineralization is not observed for subjects having chewed only gumbase, thereby clearly proving the effect of the polyols and more particularly of maltitol on this enamel remineralization.

This 6-month study perfectly demonstrates that daily consumption of maltitol has notable beneficial effects in the remineralization of dental enamel in subjects having been treated by said method. This remineralization is not due solely to the salivation factor, since the group having consumed chewing gums containing only gumbase does not show a positive remineralization effect, even though chewing promotes the salivation phenomenon.

According to one preferential mode, the active ingredient may be fluoride or any fluoride-derived compound.

According to another aspect of the present invention, a subject matter is the use of an effective amount of a polyol and more particularly maltitol and/or xylitol, preferably maltitol, for preparing a medicament intended for the treatment of dental enamel demineralization in humans or animals.

The invention will be understood more clearly from the following examples which are intended to be illustrative but nonlimiting.

### EXAMPLE

### Study of the Remineralizing Effect on Dental Enamel of a Composition Containing a Polyol

### Method of Diagnosis

The QLF™ (Quantitative Light-induced Fluorescence) method is a dental diagnostic tool for the quantitative evaluation, in vivo and in vitro, of dental carious lesions.

The apparatus is equipped with a camera and is connected to a computer (using a program specific to QLF™). The probe, which emits a blue light (xenon lamp+filter), is passed over the tooth tested, which retransmits this light in the form of fluorescence. Simultaneously, the intra-oral camera enables a photograph to be taken, which appears on the screen: it shows the differential fluorescences between the healthy tissues and the carious lesion of the tooth tested. This photograph or instantaneous image is then processed using the software of the QLF™ which then recreates a reconstituted image representing the carious lesion.

With this method, the real-time fluorescent images are entered into the computer and stored in an image database. Quantitative analysis of the images allows the user to quantify parameters such as loss of minerals, lesion depth, lesion size, size of the spots present on the teeth and seriousness of the various lesions with great precision and repeatability. The QLF™ method is based on the autofluorescence of the teeth. When the teeth are illuminated by the high-intensity blue light, they will begin to emit light in the green part of the spectrum. The intensity of the fluorescence of the dental material is directly related to the mineral content of the enamel.

Since the QLF™ method uses fluorescence to reveal the dental carie, the contrast between the demineralized enamel and the healthy enamel is increased. Thus, the absence of specular reflection in the images obtained by the QLF™ method facilitates the calculation of the size and seriousness of the lesion by the digital image processing system.

Evaluation of the efficacy against caries of a sugar-free chewing gum containing maltitol sold by the applicant under the brand SweetPearl™ using a study model consisting of various appropriate control groups (gum and maltitol, gum and xylitol, gum without polyol and no chewing gum) and the QLF™ method.

### Materials and Methods

Population Selected

Children from 8 to 13 years old, in good health, were included in the study. Their parents were informed about the study by the teachers of the school and signed an informed consent form. The children selected exhibited early carious lesions.

More specifically, 1228 pupils from 8 to 13 years old, from 12 different primary schools, were examined, and 482 pupils exhibiting the selection criteria were retained for the study. In total, 420 pupils completed a six-month study.

Study Design

This study is a double-blind (subjects, caregivers and examiners), multicenter, parallel, random clinical study carried out in schools.

Examinations

The evaluation of the dental plaque uses the Silness method and the Loe index. The digital images were taken using a digital photographic apparatus, and those of the QLF™ method were taken using the QLF™ apparatus separately. Before this examination, all the dental plaque was removed from the surface of the teeth. All the procedures were supervised by qualified personnel (dentists/hygienists). The QLF™ method was used to monitor the evolution of demineralization and remineralization of the enamel lesions. During the visit, fluorescent images were taken of the six (or fewer) anterior maxillary teeth. An image of each tooth was taken at each visit in order to ensure that all the anterior teeth were photographed. All the images taken using the QLF™ apparatus were analyzed by an experienced individual in order to determine the “mineral condition” of each tooth, using proprietory software (QLF 2.00, Inspektor, The Netherlands). The QLF™ method makes it possible to control three distinct parameters: ΔF, the area of the lesion and ΔQ. ΔF is the percentage loss of fluorescence between the comparison of a healthy enamel and an identified lesion. The detection threshold for ΔF is 5%, which means that a loss of fluorescence below this threshold is considered to be background noise and therefore not significant. The area of the lesion is calculated by means of the pixels taken into account by the analysis software as representing the demineralized enamel (i.e. those with a loss of fluorescence of at least 5%). ΔQ is the product of ΔF multiplied by the area of the lesion and represents the volume of this lesion. The main result of this study is the modification of ΔQ at a detection threshold of 5% after three, six and nine months of use of the product.

Fluorescent images were taken of the anterior maxillary teeth at the start and after 3 and 6 months.

Test Products and Consumption

All the chewing gums used in this study were produced by the applicant company according to conventional chewing gum production protocols known to those skilled in the art. They are not products available on the market.

Three treatment groups and one control placebo group were formed as indicated below:
1. “Maltitol” group (2 chewing gums containing up to 60% of maltitol to be chewed 5 times per day corresponding to a total of 10 chewing gums per day and per child).
2. “Xylitol” group (2 chewing gums containing up to 60% of xylitol to be chewed 5 times per day corresponding to a total of 10 chewing gums per day and per child).
3. “Gumbase” group (1 chewing gum to be chewed 5 times per day corresponding to a total of 5 chewing gums per day and per child).
4. “No chewing gum” group (or control).

The subjects were then responsible for chewing the chewing gums (or not) five times per day at well-defined moments in the day: after breakfast, 2 hours before lunch, after lunch, 3 hours before dinner and after dinner, and for ten minutes each time.

Statistical Methods

All the data were processed using the SPSS software, version 17.0, from the company Computer Software (SPSS Inc, Chicago, Ill., USA). The Silness index and the Loe index were noted and categorized before being evaluated using chi-squared tests. The bacterial count data, the salivary pH measurement data and the QLF™ and Nyvad parameters were subjected to an analysis of variance or Student's t test. A P value of less than 0.05 was considered to be statistically significant.

### Results

QLF™ analysis method

The results of analysis using the QLF™ method halfway through the study (after 3 months of tests) and at the end of the study (after 6 months of tests) are presented in table 1.

TABLE 1
result of the QLF ™ analysis

| QLF ™ | | | | | |
|---|---|---|---|---|---|
| parameters | Group | Sample size | Baseline | 3 Months | 6 Months |
| Area | Maltitol | 107 | 2.47 ± 1.95 | 2.33 ± 1.90 | 2.18 ± 1.63 |
| | Xylitol | 108 | 2.58 ± 1.91 | 2.43 ± 1.72 | 2.31 ± 1.85 |
| | Gum | 97 | 2.47 ± 1.70 | 2.39 ± 1.76 | 2.41 ± 1.79 |
| | No Gum | 108 | 2.41 ± 2.31 | 2.50 ± 1.85 | 2.53 ± 1.87 |
| ΔF | Maltitol | 107 | −9.38 ± 1.79 | −8.78 ± 1.78 | −8.42 ± 1.87 |
| | Xylitol | 108 | −9.36 ± 1.92 | −8.56 ± 2.02 | −8.37 ± 1.78 |
| | Gum | 97 | −9.52 ± 1.96 | −9.83 ± 2.16 | −9.9 ± 2.23 |
| | No Gum | 108 | −9.39 ± 1.98 | −9.48 ± 1.66 | −9.59 ± 2.19 |
| ΔQ | Maltitol | 107 | −24.87 ± 23.06 | −20.62 ± 18.97 | −16.69 ± 15.15 |
| | Xylitol | 108 | −25.95 ± 24.66 | −22.10 ± 18.85 | −18.19 ± 21.29 |
| | Gum | 97 | −24.71 ± 18.11 | −24.13 ± 19.74 | −24.28 ± 24.43 |
| | No Gum | 108 | −23.70 ± 22.76 | −23.50 ± 25.74 | −22.95 ± 18.90 |

The one-way AVONA analyses showed that there were no differences between the four groups at the basal level (beginning of the study) for the 3 parameters measured (area of the lesion, ΔF, ΔQ).

After 3 months of experimentation, the ΔF parameter for the maltitol group and for the xylitol group is significantly different than the ΔF parameter in the gumbase group and in the control group without gum (P<0.01). However, no significant difference was observed between the maltitol group and the xylitol group, or between the base group and the control group without gum.

After 6 months of experimentation, considerable differences exist between the ΔF and ΔQ parameters for the two treatment groups (maltitol group and xylitol group) compared with the two control groups (gumbase and no gum, p<0.01).

Thus, two of the three parameters measured (ΔF and ΔQ) significantly decreased in the two experimental groups (those having consumed maltitol and xylitol). This means that the primary caries in the enamel (demineralization) observed on the surfaces of the anterior teeth at the beginning of the study remineralized after the chewing for 6 months of chewing gums containing polyols. This remineralization is not observed for the subjects having chewed only gumbase, thereby clearly proving the effect of the polyols on this enamel remineralization. The difference is not significant between the maltitol group and the xylitol group. This means that these two polyols have a similar positive remineralization effect on the previously demineralized enamel.

The difference between the two control groups (gumbase alone and no gumbase) is not significant either, which means that the chewing gums containing only gum have no significant effect on enamel remineralization.

In conclusion, the two experimental groups (those having consumed maltitol and xylitol) are significantly more effective in stopping and reversing the oral white lesions than the two control groups.

This means that polyols, and in particular maltitol just like xylitol, clearly possess enamel-remineralizing properties, contrary to the controls.

The anti-carie effects of maltitol and xylitol are similar.

This 6-month study perfectly demonstrates that the daily consumption of polyols, and in this specific case of maltitol or of xylitol, has notable beneficial effects in the remineralization of dental enamel in primary school children.

This remineralization is not due only to the salivation factor, since the group having consumed chewing gums containing only gumbase does not show any positive remineralization effect, even though chewing promotes the salivation phenomenon.

1. A method for treating or preventing dental enamel demineralization comprising administering to a subject in need there of a composition comprising at least one polyol chosen from maltitol and xylitol.

2. The method as claimed in claim 1, wherein the composition comprising at least one polyol treats dental enamel demineralization.

3. The method as claimed in claim 1, wherein the at least one polyol is maltitol.

4. The method as claimed in claim 1, wherein the composition is administered to a human or an animal in need thereof in a proportion of from 0.05 to 3 g/kg/day.

5. The method as claimed in claim 1, wherein the composition is in the form of a sugar-free confectionery.

6. The method as claimed in claim 5, wherein the sugar-free confectionery is selected from the group consisting of hard boiled candies, dragees, jelly candies, gums, caramels, toffees and fudges, tablets, lozenges, marshmallows, chewing gums, bubble gums and chewy pastes.

7. The method as claimed in claim 6, wherein the sugar-free confectionery is a sugar-free chewing gum.

8. The method as claimed in claim 5, wherein the sugar-free confectionery is administered in a proportion of from 3 to 5 daily intakes of two confectioneries per intake, each confectionery containing between 0.5 and 1 g of said at least one polyol.

9. The method as claimed in claim 4, wherein the composition is administered for 3 months.
